(19) United States
(12) Patent Application Publication
(10) Pub. No.: US 2020/0113856 A1
(43) Pub. Date: Apr. 16, 2020

(54) NON-PRESCRIPTION MOTION SICKNESS MEDICATION CONTAINING AN ANALGESIC, AN H2 BLOCKER, AND AT LEAST ONE ANTACID

(71) Applicant: Spencer B. Jones, Billings, MT (US)

(72) Inventor: Spencer B. Jones, Billings, MT (US)

(21) Appl. No.: 16/162,362

(22) Filed: Oct. 16, 2018

(51) Int. Cl. A61K 31/192; A61K 31/167; A61K 31/616; A61P 1/08

(52) U.S. Cl. CPC A61K 31/192; A61K 31/167; A61K 36/534; A61P 1/08; A61K 2300/00; A61K 31/616

(57) ABSTRACT

The present invention provides a compounded non-prescription medication for the treatment of motion sickness. The medication comprises: at least one analgesic selected from the group consisting of acetaminophen, ibuprofen, naproxen and salicylates; at least one histamine H₂-receptor antagonist selected from the group consisting of nizatidine, famotidine, cimetidine, ranitidine. famotidine; and at least one antacid selected from the group consisting of calcium carbonate, sodium bicarbonate, magnesium hydroxide, aluminum hydroxide, and bismuth subsalicylate. The medication may optionally comprise at least one additional ingredient selected from the group consisting of spearmint extract and ginger extract. The formulations are consistent in their effectiveness and devoid of the side effects of drowsiness and dry mouth. A presently preferred embodiment of the medication comprises primarily of ibuprofen, famotidine, calcium carbonate, magnesium hydroxide, spearmint extract and ginger extract.

### BACKGROUND OF THE INVENTION

### 1. Field of the Invention

The present invention relates, generally, to medications for the relief of motion sickness and, more particularly, to over-the-counter medications for the relief of motion sickness.

### 2. History of the Prior Art

The ancient Greeks and Romans were very familiar with the phenomenon of motion sickness. In fact, the word the Greek word for seasickness, nausia or nautia, came from their word for ship, naus. But nautia or nausia also meant the worst symptom of seasickness, an upset stomach and the urge to vomit. The Romans borrowed the Greek word, spelling it nausea in Latin. If the motion causing nausea is not resolved, the sufferer will usually vomit. Vomiting often will not relieve the feeling of weakness and nausea, which means the person might continue to vomit until either the cause of the nausea is terminated or the symptoms are treated.

Motion sickness, which, depending on the cause, can also be referred to as seasickness, car sickness, simulation sickness or airsickness, occurs when there are conflicts among the senses. If, for example, you are being taken for a ride in an acrobatic aircraft and you are spinning around and repeatedly upside down, your eyes see one thing, your muscles feel another, and your inner ears sense something else. The human brain is incapable of resolving all of those mixed signals. That is why we end up feeling dizzy and sick.

The inner ears provide a sense of balance. They are part of a network called the vestibular system. This system includes three pairs of semicircular canals and two sacs, called the saccule and the utricle. They sense movement and send this information to the brain. The semicircular canals hold a fluid that moves when the head turns. The saccule and utricle are sensitive to gravity. They tell the brain whether an individual is standing up or lying down. The brain processes all of this data, and it usually comes together and makes sense. But sometimes the brain receives confusing signals.

When flying on an airliner, for example, a passenger senses movement, but his eyes tell his brain that he doesn't seem to be going anywhere. The opposite can occur, as well. After a long sea voyage, a disembarked passenger can be standing on terra firma and still feel like he is moving. The result in both cases is the same: motion sickness.

Anyone can get motion sickness, but it's most common in children and pregnant women. Motion sickness can strike quickly and, when it does, the individual typically breaks out into a cold sweat, becomes pale, feels nauseous and dizzy, notices an increase in saliva production, and loses his appetite. Some people also get headaches, feel fatigues, and experience shallow breathing.

For most people, symptoms fortunately usually don't last long. They often go away once a person becomes acclimated to the situation, whether it's the rocking of a boat or the movement of a train.

There are some simple things that one can do to relieve motion sickness if it persists. The person should try to relax, avoid reading, take deep breaths of fresh air, close the eyes, and look at a stable object. If on a boat, look at the horizon; if in a motor vehicle, look through the windshield. If flying, get a seat over the wing, where movement is the least. An upper-deck cabin can help if sailing, as can a front-seat position if sailing, or a front-seat spot if in a motor vehicle.

Natural Remedies have long been used to treat motion sickness. There is some scientific proof that raw ginger root, long used as a folk remedy to fight nausea, is effective in treating motion sickness. However, as it may act as a blood thinner, it should not be taken without first consulting a physician. Eating peppermint may also help by acting as a soothing and calming agent.

When self help and natural remedies fail to provide relief from motion sickness, there are medications which can be taken. They are usually helpful. Dimenhydrinate (Dramamine) is an over-the-counter antihistamine that is used to ease allergies. The compound also helps with balance. The first dose should be taken about an hour before traveling. More doses are taken every 4 to 6 hours. Scopolamine (Maldemar) is a prescription medication. A patch is worn behind the ear. It is typically applied about four hours before being subjected to conditions that are conducive to the onset of motion sickness. That patch is effective for three days. Other prescription drugs include cyclizine hydrochloride (Marezine, Marzine, Emoquil), meclizine (Antivert, Bonine) and promethazine (Phenadoz, Phenergan, Promethegan). These three drugs have side effects, which include drowsiness and dry mouth.

A most fascinating hypothesis for the cause of motion sickness is that it functions as a defense mechanism against the ingestion of neurotoxins. The area postrema in the brain is responsible for inducing vomiting when poisons are detected, and for resolving conflicts between vision and balance. When feeling motion but not seeing it (for example, in a ship with no windows), the inner ear transmits to the brain that it senses motion, but the eyes tell the brain that everything is still. As a result of the discordance, the brain will come to the conclusion that the individual is hallucinating and further conclude that the hallucination is due to poison ingestion. The brain responds by inducing vomiting, to clear the supposed toxin.

As astronauts frequently have motion sickness, NASA has done extensive research on the causes and treatments for motion sickness. One very promising looking treatment is for the person suffering from motion sickness to wear LCD shutter glasses that create a stroboscopic vision of 4 Hz with a dwell of 10 milliseconds.

### SUMMARY OF THE INVENTION

The present invention provides a compounded non-prescription medication for the treatment of motion sickness. The medication comprises: at least one analgesic selected from the group consisting of acetaminophen, ibuprofen, naproxen and salicylates; at least one histamine H₂-receptor antagonist selected from the group consisting of nizatidine, famotidine, cimetidine, ranitidine and roxatidine; and at least one antacid selected from the group consisting of calcium carbonate, sodium bicarbonate, magnesium hydroxide, aluminum hydroxide, and bismuth subsalicylate. The medication may optionally comprise at least one additional ingredient selected from the group consisting of spearmint extract and ginger extract.

There are presently four types of non-prescription analgesics that can be used with the various formulations of the new over-the-counter medication for motion sickness: ibuprofen; acetaminophen; naproxen; and aspirin. For a normal-strength dosage of the formulations, the following amounts of those four analgesics are: ibuprofen, 550 mg; acetaminophen, 450 mg; naproxen, 350 mg; and aspirin, 300 mg.

Of the five histamine H₂-receptor antagonists available worldwide that are listed above, only roxatidine has not yet been approved for use in the U.S. by the FDA. Although these antagonists have been largely surpassed in effectiveness by proton pump inhibitors (PPIs) such as omeprazole, lansoprazole, pantoprazole, rabeprazole, esomprazole and dexlansoprazole, the use of PPIs by the elderly can increase the risk for pneumonia. In addition, PPIs also reduce calcium absorption and increase the risk for osteoporosis. Given these serious side effects of PPIs, it is unlikely that H₂ blockers will ever be entirely supplanted by PPIs. With respect to the present invention, because PPIs typically take up to four days to become effective, as compared to one hour for histamine H₂-receptor antagonists, the use of PPIs in a compounded medication intended to provide rapid relief would be pointless.

Of the four histamine H₂-receptor antagonists available in the U.S., famotidine and nizatidine are presently the preferred members of that group. Although famotidine has been shown to be approximately 7.5 times more potent than either nizatidine or ranitidine, and 20 times more potent than cimetidine on an equimolar basis, headaches are a fairly common side effect of famotidine ingestion. Although nizatidine has been shown to be a more effective H2RA than famotidine as a maintenance therapy for patients with reflux esophagitis, and nizatidine rarely produces any undesirable side effects, nizatidine costs about five and a half times as much as a comparable dose of famotidine. Thus, if cost is not a major factor, nizatidine is the preferred H2RA because it is the least likely compound to cause undesirable side effects. However, if a low-cost formulation or a compact tablet is of paramount importance, then the scale tips in favor of famotidine.

For a normal-strength dosage of the motion sickness medication, the formulations include 12 mg of famotidine or 45 mg of nizatidine. Of the five antacids, calcium carbonate and magnesium hydroxide are the two preferred compounds, as sodium bicarbonate raises sodium levels in the body and may stress the circulatory system, aluminum hydroxide contains a metal that has been associated with plaque formation in the brains of those with Alheimer's disease, and significantly larger amounts of bismuth subsalicylate are required for neutralization of a given amount of hydrochloric acid than are required for the other four antacids. Thus, a normal-strength dosage of the medication is formulated with 900 mg of calcium carbonate and 175 mg of magnesium hydroxide. Additionally, one or both of the following extract amounts can be added to the formulations: 30 mg of spearmint extract; 200 mg of ginger extract. This combination of ingredients provides almost instant relief from motion sickness as well as other nausea inducing conditions such as heat stroke, pregnancy, and illness. This is accomplished with appropriate dosage usually starting at the onset of symptoms and with complete relief within 5-10 minutes. The formulations are consistent in their effectiveness and without the side effects of drowsiness and dry mouth that are associated with motion sickness medications now available commercially, and without the need to plan ahead by taking the medication at least an hour before the potentially nauseating activity occurs. Although the addition of spearmint and ginger extracts do not appear to be essential for treatment of the underlying condition, it has been noted that each of those ingredients seem to calm the stomach quickly as the other ingredients act.

For a maximum-strength formulation dosage, the amount of ibuprofen is increased to 850 mg, the amount of acetaminophen is increased to 900 mg, the amount of naproxen is increased to 550 mg, and the amount of aspirin is increased to 500 mg. Likewise, the amounts of famotidine, calcium carbonate and magnesium hydoxide are increased to 20 mg, 1200 mg and 200 mg, respectively. The optional amounts of spearmint extract and ginger extract are increased to 60 mg and 300 mg, respectively. For the maximum-strength formulation, 75 mg of nizatidine can be substituted for the 20 mg of famotidine.

For a low-strength formulation dosage, the amount of ibuprofen is decreased to 180 mg, the amount of acetaminophen is decreased to 150 mg, the amount of naproxen is decreased to 150 mg, and the amount of aspirin is decreased to 100 mg. Likewise, the amounts of famotidine, calcium carbonate and magnesium hydoxide are decreased to 7 mg, 450 mg and 100 mg, respectively. The optional amounts of spearmint extract and ginger extract are decreased to 10 mg and 100 mg, respectively. For the low-strength formulation dosage, 26 mg of nizatidine can be substituted for the 7 mg of famotidine.

### DETAILED DESCRIPTION OF THE INVENTION

The present invention provides a compounded non-prescription medication for the treatment of motion sickness. The medication comprises: at least one analgesic selected from the group consisting of acetaminophen, ibuprofen, naproxen and salicylates, in addition to famotidine, calcium carbonate, and magnesium hydroxide. The medication may optionally comprise at least one additional ingredient selected from the group consisting of spearmint extract and ginger extract. Each of these ingredients will now be examined in detail.

### Over-the-Counter Analgesic Component

Acetaminophen, ibuprofen, naproxen and salicylates are all analgesics. An analgesic is any member of the group of drugs used to achieve analgesia, or relief from pain. They are typically called pain killers. Analgesic drugs act in various ways on the peripheral and central nervous systems. They are distinct from anesthetics, which temporarily affect, and in some instances completely eliminate, sensation. The present invention is compounded using over-the-counter, non-opioid analgesics. Ibuprofen, acetaminophen, naproxen and salicylates fall into that class of analgesics. Each of these analgesics are capable of dulling the pain and feeling of malaise associated with motion sickness.

If multiple non-prescription analgesics are used in the formulation, the amount of each analgesic should be proportionately reduced. For example if two analgesics are used, and a standard dosage is considered to be in the middle of the specified range, then the amount of each analgesic should be half of that standard dosage amount. Likewise, if three analgesics are used, and the maximum dosage is considered to be at the top of the range of each, and equivalent amounts of each analgesic are to be used, then the amount of each analgesic should be one third of the amount at the top of the range. Use of multiple analgesics adds complexity to the formulation. For a preferred formulation of the medication, only a single analgesic is used.

### Ibuprofen

Ibuprofen is a nonsteroidal anti-inflammatory drug (NSAID) that is used for treating mild to moderate pain, fever, and inflammation. More specifically, ibuprofen is used primarily to treat painful menstruation, osteoarthritis, dental pain, headaches, and pain from kidney stones. It is also used for inflammatory diseases such as juvenile idiopathic arthritis and rheumatoid arthritis. It is also used for pericarditis and patent ductus arteriosus. Some individuals suffer adverse effects from ibuprofen intake, which may include nausea, dyspepsia, diarrhea, constipation, gastrointestinal ulceration/bleeding, headache, dizziness, rash, salt and fluid retention, and high blood pressure. Far less common adverse effects include esophageal ulceration, heart failure, high blood levels of potassium, kidney impairment, confusion, and bronchospasm. Ibuprofen has been known to exacerbate asthma, sometimes fatally. Ibuprofen was discovered in 1961 by Stewart Adams and initially marketed as Brufen. Although now available as a generic drug, it is also available under a number of trade names, including Advil and Motrin. It was first marketed in 1969 in the United Kingdom and in the United States in 1974. In spite of its relatively uncommon adverse side affects, ibuprofen is on the World Health Organization's List of Essential Medicines, the most effective and safe medicines needed in a health system.

In addition to its well-known roles in treating pain, fever and inflammation, the named inventor has discovered that ibuprofen can also be effective in treating motion sickness when compounded with other ingredients. The primary function of the analgesics is to dull the pain associated with motion sickness. In addition, with the exception of acetaminophen, the analgesics also have an anti-inflammatory effect. There appears to be an inflammatory component associated with motion sickness. The anti-inflammatory analgesics are intended to counteract that inflammatory component.

### Acetaminophen

Acetaminophen, also known as Paracetamol or APAP, is a medicine used to treat pain and fever. It is typically used for mild to moderate pain relief Evidence for its use to relieve fever in children is mixed. It is often sold in combination with other medications, such as in many cold medications. In combination with opioid pain medication, acetaminophen is also used for severe pain such as cancer pain and pain after surgery. It is typically used either by mouth or rectally but is also available intravenously. Effects last between two and four hours. Acetaminophen is generally safe at recommended doses. Serious skin rashes may rarely occur, and too high a dose can result in liver failure. It appears to be safe during pregnancy and when breastfeeding. In those with liver disease, it may still be used, but in lower doses. Acetaminophen is classified as a mild analgesic. It does not have significant anti-inflammatory activity and how it works is not entirely clear. Acetaminophen was first made in 1877. It is the most commonly used medication for pain and fever in both the United States and Europe. It is on the World Health Organization's List of Essential Medicines. Acetaminophen is available as a generic medication and under trademarks, which include Tylenol and Panadol.

Although ibuprofen seems to be the preferred analgesic for compounding the over-the-counter motion sickness formulation of the present invention, it is generally believed that acetaminophen is safer for use by young children than is ibuprofen. Thus, the motion sickness formulation has also been compounded using acetaminophen.

### Naproxen

Naproxen (also known by the brand names Aleve, Naprosyn, and many others) is a nonsteroidal anti-inflammatory drug (NSAID) of the propionic acid class (the same class as ibuprofen) that relieves pain, fever, swelling, and stiffness. It is a nonselective COX inhibitor, usually sold as the sodium salt, naproxen sodium. It is available in both an immediate release and extended release formulations. Naproxen is generally safe for use by breastfeeding mothers. Naproxen is used for the management of mild to moderate pain, fever, and inflammation. It works by reducing the levels of prostaglandins, chemicals that are responsible for pain, fever, and inflammation. Naproxen blocks the enzyme that makes prostaglandins (cyclooxygenase), resulting in lower concentrations of prostaglandins. As a consequence, inflammation, pain and fever are reduced. Naproxen was approved by the FDA in December 1991.

As with other non-COX-2 selective NSAIDs, naproxen can cause gastrointestinal problems, such as heartburn, constipation, diarrhea, ulcers and stomach bleeding. Naproxen should be taken orally with food to decrease the risk of gastrointestinal side effects. Persons with a history of ulcers or inflammatory bowel disease should consult a doctor before taking naproxen. In the U.S., naproxen is sold with boxed warnings about the risk of gastrointestinal ulceration or bleeding. Naproxen poses an intermediate risk of stomach ulcers compared with ibuprofen, which is low-risk, and indometacin, which is high-risk. To reduce stomach ulceration risk, it is often combined with a proton-pump inhibitor (a medication that reduces stomach acid production) during long-term treatment of those with pre-existing stomach ulcers or a history of developing stomach ulcers while on NSAIDs.

COX-2 selective and nonselective NSAIDs have been linked to increases in the number of serious and potentially fatal cardiovascular events, such as myocardial infarctions and strokes. Naproxen is, however, associated with the smallest overall cardiovascular risks. Cardiovascular risk must be considered when prescribing any nonsteroidal anti-inflammatory drug. The drug had roughly 50% of the associated risk of stroke compared with ibuprofen, and was also associated with a reduced number of myocardial infarctions.

A study found that high-dose naproxen induced near-complete suppression of platelet thromboxane throughout the dosing interval and appeared not to increase cardiovascular disease (CVD) risk, whereas other non-aspirin high-dose NSAID regimens had only transient effects on platelet COX-1 and were associated with a small but definite vascular hazard. Conversely, naproxen was associated with higher rates of upper gastrointestinal bleeding complications compared with other NSAIDs

### Salicylates

Salicylates, which include aspirin, magnesium salicylate, and sodium salicylate, constitute the final group of over-the-counter analgesics. Of the three salicylates, aspirin is. by far, the most common. Also known as acetylsalicylic acid (ASA), aspirin is a medication used to treat pain, fever, and inflammation. Specific inflammatory conditions in which aspirin is used include Kawasaki disease, pericarditis, and rheumatic fever. Aspirin given shortly after a heart attack decreases the risk of death. Aspirin is also used long-term to help prevent heart attacks, ischaemic strokes, and blood clots in people at high risk. It may also decrease the risk of certain types of cancer, particularly colorectal cancer. For pain or fever, effects typically begin within 30 minutes. Like ibuprofen, aspirin is a nonsteroidal anti-inflammatory drug and works in ways that are similar to those of other NSAIDs, but also suppresses the normal functioning of platelets.

One common adverse side effect is an upset stomach. More significant side effects include stomach ulcers, stomach bleeding, and worsening asthma. Bleeding risk is greater among those who are older, drink alcohol, take other NSAIDs, or are on other blood thinners. Aspirin is not recommended during the last trimester of pregnancy. At high dosage levels, ringing in the ears may occur. In addition, it is generally not recommended in children with infections because of the risk of Reye syndrome. Reye syndrome is a rapidly progressive encephalopathy. Symptoms may include vomiting, personality changes, confusion, seizures, and loss of consciousness. Even though liver toxicity typically occurs, yellowish skin usually does not. Death occurs in 20-40% of those affected and about a third of those who survive are left with a significant degree of brain damage. When aspirin was withdrawn for use in children a decrease of more than 90 percent in rates of Reye syndrome was seen.

Salicin, a precursor to aspirin found in leaves of the willow tree, has been used for its health effects for at least 2,400 years. Known to Western doctors since the mid-19th century, it was used sparingly due to its unpleasant taste and tendency to damage the stomach. In 1853, chemist Charles Frederic Gerhardt treated the medicine sodium salicylate with acetyl chloride to produce acetylsalicylic acid—a compound that is less damaging to the stomach—for the first time. For the next fifty years, other chemists established the chemical structure and came up with more efficient methods to make it. During the final years of the 19th century, the German company, Bayer AG, managed to patent acetylcalicylic acid. Once patented, Bayer began distributing the drug, under the trademark “Aspirin”, in powder form to physicians to give to their patients in one-gram doses. It quickly became the number-one drug worldwide. Aspirin is still one of the most widely used medications globally, with an estimated 40,000,000 kg (or up to 120 billion pills) consumed each year. It, too, is on the World Health Organization's of Essential Medicines.

Because of the risk of Reye syndrome, aspirin should not be given to persons in their teens and younger. Thus, the compounding of the motion sickness formulation using one of the salicylates as the analgesic, is intended for use for only adults who are at least twenty years of age. Although aspirin is the preferred salicylate because of its ready availability, equivalent amounts of magnesium salicylate or sodium salicylate can also be used.

### Histamine H₂-Receptor Antagonists

At least one histamine H₂-receptor antagonists (also known as H₂ blockers, histamine-2 receptor antagonists or, simply, H2RA) is another ingredient of the compounded formulation for treating motion sickness. Drugs in this class block the action of histamine at the histamine H₂ receptors of the parietal cells in the stomach. This decreases the amount of acid the stomach produces, which is why at least one H2RA is included in the compounded medication for treating motion sickness. Historically, H2RAs have been used in the treatment of dyspepsia, peptic ulcers and gastroesophageal reflux disease, and to prevent intestinal ulcers from returning after they have healed. Five H2RAs are presently available worldwide: famotidine, cimetidine, ranitidine, nizatidine and roxatidine. Of these five medications, only roxatidine has not yet been approved for use in the U.S. by the FDA. H2RAs have been largely surpassed in effectiveness by proton pump inhibitors (PPIs). However, for the elderly, use of PPIs can increase the risk for pneumonia. In addition, PPIs also reduce calcium absorption and increase the risk for osteoporosis. Given these serious side effects of PPIs, it is unlikely that H₂ blockers will be completely supplanted by PPIs. Although conceivable that, for the compounded formulation to treat motion sickness of the present invention, H2-receptor antagonists might be replaced by proton pump inhibitors, such as omeprazole, lansoprazole, pantoprazole, rabeprazole, esomprazole and dexlansoprazole, it typically takes up to four days for proton pump inhibitors to become effective. Thus, the use of a proton pump inhibitor in a medication intended to provide relief of motion sickness symptoms in less than an hour would be pointless.

Of the five available H2RAs available in the U.S., famotidine and nizatidine are presently the preferred members of that group. Although famotidine has been shown to be approximately 7.5 times more potent than ranitidine and 20 times more potent than cimetidine on an equimolar basis, headaches are a fairly common side effect of famotidine use. Although nizatidine has been shown to be a more effective H2RA than famotidine as a maintenance therapy for patients with reflux esophagitis, and nizatidine rarely produces any undesirable side effects, nizatidine costs about five and a half times as much as a comparable dose of famotidine. Thus, if cost is not a major factor, nizatidine is the preferred H2RA because it is the least likely compound to cause undesirable side effects. However, if a low-cost formulation is of paramount importance, then the scale tips in favor of famotidine.

### Antacids

The present invention is compounded using at least one antacid. Antacids are substances which neutralizes stomach acid, and are used to relieve heartburn, indigestion or an upset stomach. Antacids are available over the counter and are taken by mouth to quickly relieve occasional heartburn, the major symptom of gastroesophageal reflux disease and also indigestion. Treatment with antacids alone is symptomatic and only justified for minor symptoms. There are five commonly available antacids: calcium carbonate, sodium bicarbonate, magnesium hydroxide, aluminum hydroxide and bismuth subsalicylate. Each of these compounds will be described below.

### Calcium Carbonate

Calcium carbonate is the calcium salt (CaCO3) of carbonic acid. Essentially chalk, it is an odorless, tasteless powder or crystal that occurs in nature. Practically insoluble in water, it occurs extensive in rocks world-wide. Ground calcium carbonate results directly from the mining of limestone. Calcium carbonate is used therapeutically as a phosphate buffer in hemodialysis, as an antacid in gastric hyperacidity for temporary relief of indigestion and heartburn, and as a calcium supplement for preventing and treating osteoporosis.

### Sodium Bicarbonate

Sodium bicarbonate (IUPAC name: sodium hydrogen carbonate), commonly known as baking soda, is a chemical compound with the formula NaHCO₃. It is a salt composed of a sodium cation (Na⁺) and a bicarbonate anion (HCO3⁻). Sodium bicarbonate is a white solid that is crystalline, but is often prepared as a fine powder. Sodium bicarbonate mixed with water can be used as an antacid to treat acid indigestion and heartburn. Its reaction with stomach acid produces salt, water, and carbon dioxide:

NaHCO₃+HCl→NaCl+H₂O+CO₂(g)

Intravenous sodium bicarbonate in an aqueous solution is sometimes used for cases of acidosis, or when insufficient sodium or bicarbonate ions are in the blood. In cases of respiratory acidosis, the infused bicarbonate ion drives the carbonic acid/bicarbonate buffer of plasma to the left, and thus raises the pH. For this reason, sodium bicarbonate is used in medically supervised cardiopulmonary resuscitation.

Sodium bicarbonate has been found to have no effect on the blood pressure of several types of rat models susceptible to salt-sensitive hypertension, in contrast with sodium chloride. Hypertension was ascribed to the high concentration of chloride, rather than the sodium content in dietary salts.

### Magnesium Hydroxide

Magnesium hydroxide is an inorganic compound with the chemical formula Mg(OH)₂. It occurs in nature as the mineral brucite. It is a white solid with low solubility in water. Magnesium hydroxide is a common component of antacids, such as milk of magnesia, as well as laxatives.

### Aluminum Hydroxide

Aluminium hydroxide, Al(OH)₃, is found in nature as the mineral gibbsite (also known as hydrargillite) and its three much rarer polymorphs: bayerite, doyleite, and nordstrandite. Aluminium hydroxide is amphoteric in nature, i.e., it has both basic and acidic properties. Closely related are aluminium oxide hydroxide, AlO(OH), and aluminium oxide or alumina (Al₂O₃), the latter of which is also amphoteric. These compounds together are the major components of the aluminium ore bauxite.

Virtually all the aluminium hydroxide used commercially is manufactured by the Bayer process[9] which involves dissolving bauxite in sodium hydroxide at temperatures up to 270° C. (518° F.). The waste solid, bauxite tailings, is removed and aluminium hydroxide is precipitated from the remaining solution of sodium aluminate. This aluminium hydroxide can be converted to aluminium oxide or alumina by calcination.

Under the generic name “algeldrate”, aluminium hydroxide is used as an antacid in humans and animals (mainly cats and dogs). It is preferred over other alternatives such as sodium bicarbonate because Al(OH)3, being insoluble, does not increase the pH of stomach above 7 and hence, does not trigger secretion of excess acid by the stomach. It reacts with excess acid in the stomach, reducing the acidity of the stomach content, which may relieve the symptoms of ulcers, heartburn or dyspepsia. Such products can cause constipation, because the aluminium ions inhibit the contractions of smooth muscle cells in the gastrointestinal tract, slowing peristalsis and lengthening the time needed for stool to pass through the colon. Some such products (such as Maalox®) are formulated to minimize such effects through the inclusion of equal concentrations of magnesium hydroxide or magnesium carbonate, which have counterbalancing laxative effects.

### Bismuth Subsalicylate

Bismuth subsalicylate, sold under the brand name Pepto-Bismol®, is an antacid medication used to treat temporary discomforts of the stomach and gastrointestinal tract, such as diarrhea, indigestion, heartburn and nausea. It is also commonly known as pink bismuth.

Bismuth subsalicylate has the empirical chemical formula of C₇H₅BiO₄, and it is a colloidal substance obtained by hydrolysis of bismuth salicylate (Bi(C₆H₄(OH)CO₂)₃).

The ingestion of bismuth subsalicylates can have some adverse effects. It can cause a black tongue and black stools in some users of the drug, when it combines with trace amounts of sulfur in saliva and the colon to form bismuth sulfide. Bismuth sulfide is a highly insoluble black salt, and the discoloration seen is temporary and harmless. Long-term use (greater than 6 weeks) may lead to accumulation and toxicity. Some of the risks of salicylism can apply to the use of bismuth subsalicylate. Children should not take medication with bismuth subsalicylate while recovering from influenza or chicken pox, as epidemiologic evidence points to an association between the use of salicylate-containing medications during certain viral infections and the onset of Reye's syndrome. For the same reason, it is typically recommended that nursing mothers not use medication containing bismuth subsalicylate because small amounts of the medication are excreted in human breast milk, and these pose a theoretical risk of Reye's syndrome to nursing children.

### Plant Extracts

Certain plant extracts are notable for their anti-inflammatory characteristics and their ability to calm the body. Two such extracts, spearmint and ginger, will be described below. They are considered optional components of the compounded formulation for the treatment of motion sickness.

### Spearmint Extract

Spearmint is an herb. The leaves and oil are commonly used for medicinal purposes. Spearmint extract is a purified and concentrated form of the spearmint herb. Spearmint, like other members of the mint family, has a square shaped stem. Spearmint gets its name from the fact that its leaves are shaped like spears. It's traditionally grown around the Mediterranean, where spearmint plants can grow to be about 3 feet tall. Spearmint is ingested for conditions such as flatulence, indigestion, nausea, vomiting, and other conditions, but there seems to be no good scientific evidence to support these uses. Nevertheless, the oil in spearmint does contain chemicals that have been shown to reduce inflammation and change levels of hormones, such as testosterone, in the body. Some chemicals found in spearmint might also harm cancer cells and kill bacteria. It also has a pleasing scent that may act to calm the body.

### Ginger Extract

Ginger is a plant with leafy stems and yellowish green flowers. The ginger spice comes from the roots of the plant. In foods and beverages, ginger spice is used as a flavoring agent. Ginger is native to warmer parts of Asia, such as China, Japan, and India, but now is grown in parts of South American and Africa. It is now also grown in the Middle East for use as medicine and as a spice.

Ginger is commonly used for various types of alimentary tract problems, including motion sickness, morning sickness, colic, upset stomach, gas, diarrhea, irritable bowel syndrome, nausea, nausea caused by cancer treatment, nausea caused by HIV/AIDS treatment, nausea and vomiting after surgery, as well as loss of appetite. One of the chemicals in ginger is also used as an ingredient in laxative, anti-gas, and antacid medications.

Ginger contains chemicals that may reduce nausea and inflammation. Researchers believe the chemicals work primarily in the stomach and intestines, but they may also work in the brain and nervous system to control nausea. Ginger clearly has credentials that recommend it as an ingredient in the compounded medication.

### Low, Standard and Maximum Dosage Formulations

For a normal-strength dosage of the motion sickness medication, the formulations include 12 mg of famotidine or 45 mg of nizatidine. Of the five antacids, calcium carbonate and magnesium hydroxide are the two preferred compounds, as sodium bicarbonate raises sodium levels in the body and may stress the circulatory system, aluminum hydroxide contains a metal that has been associated with plaque formation in the brains of those with Alheimer's disease, and significantly larger amounts of bismuth subsalicylate are required for neutralization of a given amount of hydrochloric acid than are required for the other four antacids. Thus, a normal-strength dosage of the medication is formulated with 900 mg of calcium carbonate and 175 mg of magnesium hydroxide. Additionally, one or both of the following extract amounts can be added to the formulations: 30 mg of spearmint extract; 200 mg of ginger extract. This combination of ingredients provides almost instant relief from motion sickness as well as other nausea inducing conditions such as heat stroke, pregnancy, and illness. This is accomplished with appropriate dosage usually starting at the onset of symptoms and with complete relief within 5-10 minutes. The formulations are consistent in their effectiveness and without the side effects of drowsiness and dry mouth that are associated with motion sickness medications now available commercially, and without the need to plan ahead by taking the medication at least an hour before the potentially nauseating activity occurs. Although the addition of spearmint and ginger extracts do not appear to be essential for treatment of the underlying condition, it has been noted that each of those ingredients seem to calm the stomach quickly as the other ingredients act.

For a maximum-strength formulation dosage, the amount of ibuprofen is increased to 850 mg, the amount of acetaminophen is increased to 900 mg, the amount of naproxen is increased to 550 mg, and the amount of aspirin is increased to 500 mg. Likewise, the amounts of famotidine, calcium carbonate and magnesium hydoxide are increased to 20 mg, 1200 mg and 200 mg, respectively. The optional amounts of spearmint extract and ginger extract are increased to 60 mg and 300 mg, respectively. For the maximum-strength formulation, 75 mg of nizatidine can be substituted for the 20 mg of famotidine.

For a low-strength formulation dosage, the amount of ibuprofen is decreased to 180 mg, the amount of acetaminophen is decreased to 150 mg, the amount of naproxen is decreased to 150 mg, and the amount of aspirin is decreased to 100 mg. Likewise, the amounts of famotidine, calcium carbonate and magnesium hydoxide are decreased to 7 mg, 450 mg and 100 mg, respectively. The optional amounts of spearmint extract and ginger extract are decreased to 10 mg and 100 mg, respectively. For the low-strength formulation dosage, 26 mg of nizatidine can be substituted for the 7 mg of famotidine.

### Reduction of Stomach Acid May be the Key to Effectiveness of the Formulations

By essentially eliminating acid in the stomach through the use of either famotidine or nizatidine (which suppress its formation) and of calcium carbonate and magnesium hydroxide (which both act to neutralize the acid that is already within the stomach), the brain may be sent signals by the digestive system that leads it to conclude that there is nothing in the stomach to expel.

### Override of Communications Between Brain, Vestibular and Digestive Systems May be Key

An alternative theory is that the symptoms of motion sickness, heat sickness, and nausea are a caused by dysfunction of the complex communications between the brain and the vestibular and digestive systems. Once communication to one of these systems gets disrupted it not only causes the associated symptoms, as that system turns to chaos, but also causes a chain reaction affecting the other system. The simultaneous ingestion of famotidine, calcium carbonate and magnesium hydroxide may cause an override of the mixed signals that are being transmitted between the brain and the digestive system. The analgesic may cause an override of the signals between the brain and the vestibular system. This is why the combination is so essential, because of the chain reaction that takes place.

### CONCLUSION

Ockham's razor is the problem-solving maxim that the simplest of competing hypotheses is more likely to be the correct one. The principle is attributed to William of Ockham (c. 1287-1347), who was an English Franciscan friar, scholastic philosopher, and theologian. If Ockham's razor is applied to the previous two hypotheses, the simpler first hypothesis is the favored explanation. However, neither hypothesis has been tested or researched. Thus, neither may ultimately prove to be correct and a third explanation may win out.

What is claimed is:

1. A compounded non-prescription medication for the treatment of motion sickness comprising:
at least one analgesic selected from the group consisting of ibuprofen, acetaminophen, naproxen and salicylates;
at least one histamine H₂-receptor antagonist; and
at least one antacid.

2. (canceled)

3. The compounded non-prescription medication of claim 1, wherein said at least one antacid is selected from the group consisting of calcium carbonate, sodium bicarbonate, magnesium hydroxide, aluminum hydroxide, and bismuth subsalicylate.

4. The compounded non-prescription medication of claim 1, wherein said at least one histamine H₂-receptor antagonist consists primarily of famotidine, and said at least one antacid consists primarily of calcium carbonate and magnesium hydroxide.

5. (canceled)

6. The compounded non-prescription medication of claim 4, wherein the analgesic is primarily ibuprofen having a mass within a range of about 180 mg to about 850 mg, famotidine has a mass within a range of about 7 mg to about 20 mg, calcium carbonate has a mass within a range of about 450 mg to about 1200 mg, and magnesium hydroxide has a mass within a range of about 100 mg to about 200 mg.

7. The compounded non-prescription medication of claim 4, which further comprises:
at least one additional ingredient selected from the group consisting of spearmint extract and ginger extract; and
wherein spearmint extract, when incorporated, has a mass within a range of about 10 mg to about 60 mg; and
wherein ginger extract, when incorporated, has a mass within a range of about 100 mg to about 300 mg.

8. The compounded non-prescription medication of claim 4, wherein:
the analgesic is primarily acetaminophen having a mass within a range of about 150 mg to about 900 mg;
famotidine has a mass within a range of about 7 mg to about 20 mg;
calcium carbonate has a mass within a range of about 450 mg to about 1200 mg; and
magnesium hydroxide has a mass within a range of about 100 mg to about 200 mg.

9. The compounded non-prescription medication of claim 8, which further comprises:
at least one additional ingredient selected from the group consisting of spearmint extract and ginger extract; and
wherein spearmint extract, when incorporated, has a mass within a range of about 10 mg to about 60 mg; and
wherein ginger extract, when incorporated, has a mass within a range of about 100 mg to about 300 mg.

10. The compounded non-prescription medication of claim 4, wherein:
the alalgesic is primarily naproxen having a mass within a range of about 150 mg to about 550 mg;
famotidine has a mass within a range of about 7 mg to about 20 mg;
calcium carbonate has a mass within a range of about 450 mg to about 1200 mg; and
magnesium hydroxide has a mass within a range of about 100 mg to about 200 mg.

11. The compounded non-prescription medication of claim 10, which further comprises:
at least one additional ingredient selected from the group consisting of spearmint extract and ginger extract; and
wherein spearmint extract, when incorporated, has a mass within a range of about 10 mg to about 60 mg; and
wherein ginger extract, when incorporated, has a mass within a range of about 100 mg to about 300 mg.

12. The compounded non-prescription medication of claim 4, wherein:
the analgesic is primarily the salicylate, aspirin, having a mass within a range of about 100 mg to about 500 mg;
famotidine has a mass within a range of about 7 mg to about 20 mg;
calcium carbonate has a mass within a range of about 450 mg to about 1200 mg; and
magnesium hydroxide has a mass within a range of about 100 mg to about 200 mg.

13. The compounded non-prescription medication of claim 12, which further comprises:
at least one additional ingredient selected from the group consisting of spearmint extract and ginger extract; and
wherein spearmint extract, when incorporated, has a mass within a range of about 10 mg to about 60 mg; and
wherein ginger extract, when incorporated, has a mass within a range of about 100 mg to about 300 mg.

14. A compounded non-prescription medication for the treatment of motion sickness comprising:
a single analgesic selected from the group consisting of ibuprofen, acetaminophen, naproxen and salicylates;
at least one histamine H₂-receptor antagonist; and
at least one antacid selected from the group consisting of calcium carbonate, sodium bicarbonate, magnesium hydroxide, aluminum hydroxide, and bismuth subsalicylate.

15. The compounded non-prescription medication of claim 14, wherein the masses of each of the analgesics ibuprofen, acetaminophen, naproxen and aspirin fall within ranges of about 180 mg to about 850 mg, about 150 mg to about 900 mg, about 150 mg to about 550 mg, and about 100 mg to 500 mg, respectively.

16. The compounded non-prescription medication of claim 15, wherein said at least one histamine H₂-receptor antagonist consists primarily of famotidine, and said at least one antacid consists primarily of calcium carbonate and magnesium hydroxide.

17. The compounded non-prescription medication of claim 16, wherein:
famotidine has a mass within a range of about 7 mg to about 20 mg;
calcium carbonate has a mass within a range of about 450 mg to about 1200 mg; and
magnesium hydroxide has a mass within a range of about 100 mg to about 200 mg.

18. The compounded non-prescription medication of claim 17, which further comprises:
at least one additional ingredient selected from the group consisting of spearmint extract and ginger extract; and
wherein spearmint extract, when incorporated, has a mass within a range of about 10 mg to about 60 mg; and
wherein ginger extract, when incorporated, has a mass within a range of about 100 mg to about 300 mg.

19. A compounded non-prescription medication for the treatment of motion sickness comprising:
at least one analgesic selected from the group consisting of ibuprofen, acetaminophen, naproxen and salicylates;
famotidine, within a range of about 7 mg to about 20 mg;
calcium carbonate, within a range of about 450 mg to about 1200 mg;
magnesium hydroxide, within a range of about 100 mg to about 200 mg;
spearmint extract within a range of about 0 mg to about 60 mg; and
ginger extract within a range of about 0 mg to about 300 mg;
wherein the amounts of ibuprofen, acetaminophen, naproxen and aspirin fall within ranges of about 180 mg to about 850 mg, about 150 mg to about 900 mg, about 150 mg to about 550 mg, and about 100 mg to 500 mg, respectively; and
wherein when multiple analgesics are used, each is reduced proportionately.

20. The compounded non-prescription medication of claim 19, wherein said at least one analgesic excludes salicylates when formulated for those less than twenty years of age.
